(19) United States
(12) Patent Application Publication
(10) Pub. No.: US 2008/0004443 A1
(43) Pub. Date: Jan. 3, 2008

(54) Sulfonated polyaryletherketone-block-polyethersulfone copolymers

(75) Inventors: Daniel Joseph Brunelle, Burnt Hills, NY (US); Hongyi Zhou, Niskayuna, NY (US); Hongwei Liu, Schenectady, NY (US); Joyce Hung, Niskayuna, NY (US); Marianne Elisabeth Harmon, Niskayuna, NY (US); David Roger Moore, Albany, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/479,202

(22) Filed: Jul. 3, 2006

(51) Int. Cl. C07D 295/00

(52) U.S. Cl. 544/162

(57) ABSTRACT

Sulfonated block copolymer suitable for use as proton exchange membranes for fuel cells comprise sulfonated polyaryletherketone blocks and polyethersulfone blocks. The sulfonated polyaryletherketone blocks comprise structural units of formula I

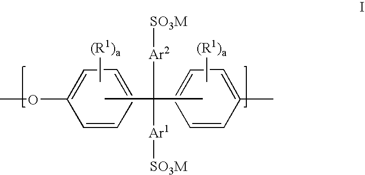

wherein R¹ is C₁-C₁₀ alkyl, C₃-C₁₂ cycloalkyl, C₆-C₁₄ aryl, allyl, alkenyl, alkoxy, halo, or cyano;
Ar¹ and Ar² are each independently C₆-C₂₀ aromatic radicals, or Ar¹ and Ar², taken together with an intervening carbon atom, form a bicyclic C₆-C₂₀ aromatic radical or a tricyclic C₆-C₂₀ aromatic radical;
M is H, a metal cation, a non-metallic inorganic cation, an organic cation or a mixture thereof; and
a is 0 or an integer from 1 to 4.

### BACKGROUND

The invention relates generally to sulfonated polyaryletherketone-block-polyethersulfone copolymers for use as proton exchange membranes.

Interest in using fuel cells as a clean, alternative power source has driven years of intense research in polymer electrolyte membrane (PEM) fuel cell development to meet the cost and performance targets for automotive and portable applications. Current PEM fuel cells use mainly Nafion® or other perfluorosulfonic acid polymer membranes which have high proton conductivity and good chemical and mechanical stability under fully humidified conditions. However, the widespread use of these membranes has been limited by their high cost and poor performance at low relative humidities (RH). Therefore, alternative low-cost membrane materials which have better performance in less humidified conditions are desired.

Both polyethersulfones (PES) and polyaryletherketones (PAEK) such as polyetheretherketones (PEEK) are known for their excellent chemical and mechanical properties. The presence of crystallinity in PAEK also imparts solvent resistance. Sulfonated PES and PAEK polymers have been studied extensively for PEM fuel cell membrane applications. Polyaryletherketones are easily sulfonated by treatment with concentrated sulfuric acid. Therefore sulfonated PAEK (SPAEK) polymers, particularly sulfonated polyetheretherketones (SPEEK), reported to date have mostly been synthesized by post-sulfonation. However, directly copolymerized SPEEK polymers have also been reported recently. While polymer blends of SPEEK/PES have been described (Manea, et al., J. Membr. Sci., 206, 443-453 (2002)), block copolymers of SPEEK and PES have not been reported.

### BRIEF DESCRIPTION

It has been unexpectedly discovered that sulfonated polyaryletherketone-unsulfonated polyethersulfone block copolymers exhibit proton conductivities better than Nafion® 117 at 80° C., 25% relative humidity (RH). The block copolymers are expected to have increased phase separation between the hydrophilic and hydrophobic domains, resulting in a more open and connected ionic network for proton conduction. These polymers are suitable for replacing Nafion® in fuel cells for high temperature, low humidity applications.

Accordingly, in one aspect, the present invention relates to sulfonated block copolymers comprising sulfonated polyaryletherketone blocks and unsulfonated polyethersulfone blocks. The sulfonated polyaryletherketone blocks comprise structural units of formula I

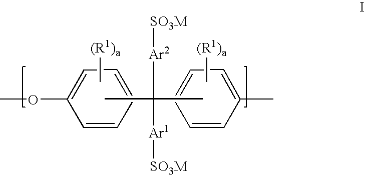

wherein R¹ is C₁-C₁₀ alkyl, C₃-C₁₂ cycloalkyl, C₆-C₁₄ aryl, allyl, alkenyl, alkoxy, halo, or cyano;

### Ar¹ and Ar² are each independently C₆-C₂₀ aromatic radicals, or Ar¹ and Ar², taken together with an intervening carbon atom, form a bicyclic C₆-C₂₀ aromatic radical or a tricyclic C₆-C₂₀ aromatic radical;

### M is H, a metal cation, a non-metallic inorganic cation, an organic cation or a mixture thereof; and

a is 0 or an integer from 1 to 4.

In another aspect, the present invention relates to proton exchange membranes comprising the sulfonated block copolymers according to the present invention, and fuel cells containing them.

### DRAWINGS

These and other features, aspects, and advantages of the present invention will become better understood when the following detailed description is read with reference to the accompanying drawings in which like characters represent like parts throughout the drawings, wherein:

FIG. 1 Comparison of conductivity of Nafion® 117 and polymer HL-3590-41 at 80° C. at different relative humidities.

### DETAILED DESCRIPTION

In one embodiment, the present invention relates to sulfonated block copolymers comprising sulfonated polyaryletherketone blocks and unsulfonated polyethersulfone blocks. The sulfonated polyaryletherketone blocks comprise structural units of formula I

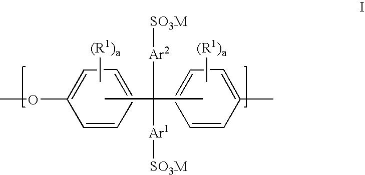

wherein R¹ is C₁-C₁₀ alkyl, C₃-C₁₂ cycloalkyl, C₆-C₁₄ aryl, allyl, alkenyl, alkoxy, halo, or cyano;

### Ar¹ and Ar² are each independently C₆-C₂₀ aromatic radicals, or Ar¹ and Ar², taken together with an intervening carbon atom, form a bicyclic C₆-C₂₀ aromatic radical or a tricyclic C₆-C₂₀ aromatic radical;

### M is H, a metal cation, a non-metallic inorganic cation, an organic cation or a mixture thereof; and

a is 0 or an integer from 1 to 4.

The sulfonated polyaryletherketone blocks further comprise structural units of formula II

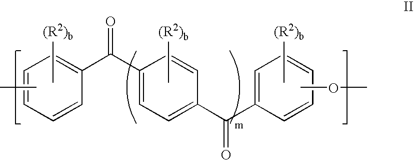

wherein R² is C₁-C₁₀ alkyl, C₃-C₁₂ cycloalkyl, C₆-C₁₄ aryl, allyl, alkenyl, alkoxy, halo, or cyano;
b is 0 or an integer from 1 to 4; and
m is 0 or 1.

The unsulfonated polyethersulfone blocks comprise structural units of formula III

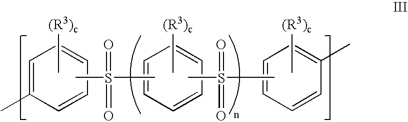

wherein R³ is C₁-C₁₀ alkyl, C₃-C₁₂ cycloalkyl, C₆-C₁₄ aryl, allyl, alkenyl, alkoxy, halo, or cyano;
c is 0 or an integer from 1 to 4; and
n is 0 or 1.

The unsulfonated polyethersulfone blocks further comprise structural units of formula IV

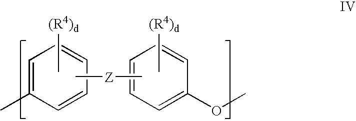

wherein R⁴ is C₁-C₁₀ alkyl, C₃-C₁₂ cycloalkyl, C₆-C₁₄ aryl, allyl, alkenyl, alkoxy, halo, or cyano;

### Z is a direct bond, O, S, (CH₂)ᵣ, (CF₂)ᵣ, C(CH₃)₂, C(CF₃)₂, or SO₂;

d is 0 or an integer from 1 to 4; and
r is an integer from 1 to 5.

In another embodiment, the invention relates to a sulfonated block copolymer comprising sulfonated polyaryletherketone blocks comprising structural units of formula V

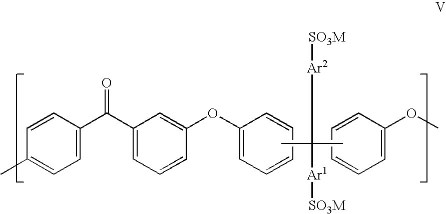

and unsulfonated polyethersulfone blocks comprising structural units of formula VI

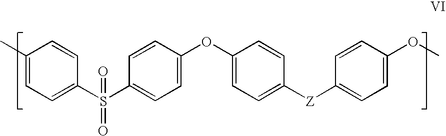

### Ar¹ and Ar² are each independently C₆-C₂₀ aromatic radicals, or Ar¹ and Ar², taken together with an intervening carbon atom, form a bicyclic C₆-C₂₀ aromatic radical or a tricyclic C₆-C₂₀ aromatic radical;

### M is H, a metal cation, a non-metallic inorganic cation, an organic cation or a mixture thereof;

### Z is a direct bond, O, S, (CH₂)ᵣ, (CF₂)ᵣ, C(CH₃)₂, C(CF₃)₂, or SO₂;

r is an integer from 1 to 5.

In particular embodiments, any of a, b, c, or d may be 0. In specific embodiments, all of a, b, c, or d are 0, and the block copolymer is composed of unsubstituted structural units, except for the sulfonate groups.

In other embodiments, Z is a direct bond, and the block copolymer is composed of structural units derived from biphenol; in still other embodiments, Z is a C(CF₃)₂, and the block copolymer is composed of structural units derived from 4,4′-(hexafluoroisopropylidene) diphenol; and in yet other embodiments, Z is SO₂, and the block copolymer is composed of structural units derived from bis(4-hydroxyphenyl) sulfone.

In some embodiments, structural units of formula I are

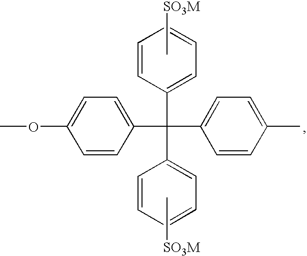

and the block copolymer is composed of structural units derived from 4,4′-dihydroxytetraphenylmethane; and in still other embodiments, the structural units of formula I are

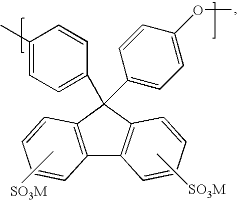

and the block copolymer is composed of structural units derived from 9,9-bis(4-hydroxyphenyl) fluorene.

The structural units of formula II may be derived from aromatic dihalo compounds. Exemplary aromatic dihalo compounds include, but not limited to, 4,4′-dichlorobenzophenone and 4,4′-difluorobenzophenone, 1,4-bis(4-fluorobenzoyl)benzene, 1,3-bis(4-fluorobenzoyl)benzene, 1,4-bis(4-chlorobenzoyl)benzene, and the like.

The structural units of formula III may be derived from one or more dihydroxyaryl monomers, particularly bisphenol monomers. Exemplary dihydroxy monomers useful in the invention include, but not limited to, 4,4′-dihydroxydiphenyl sulfone, 4,4′-(hexafluoroisopropylidene) diphenol, and the like. Additional diphenols may also be added to the reaction mixture to form the block copolymers. The structural units of formula I may be derived from one or more dihydroxyaryl monomers, particularly bisphenol monomers. Exemplary dihydroxy monomers useful in the invention include, but not limited to, 4,4′-dihydroxytetraphenylmethane, 9,9-bis(4-hydroxyphenyl) fluorene, 4,4′-(hexafluoroisopropylidene) diphenol, and the like. Other dihydroxyaryl monomers that may be used to prepare the unsulfonated polyarylethersulfones include 1,1-bis-(4-hydroxyphenyl)cyclopentane; 2,2-3-allyl-4-hydroxyphenyl) propane; 2,2-bis-(2-t-butyl-4-hydroxy-5-methylphenyl) propane; 2,2-bis-(3-t-butyl-4-hydroxy-6-methylphenyl) propane; 2,2-bis-(3-t-butyl-4-hydroxy-6-methylphenyl) butane; 2,2-bis-(3-methyl-4-hydroxyphenyl) propane; 1,1-4-hydroxy-phenyl)-2,2,2-trichloroethane; 1,1-bis-(4-hydroxyphenyl) norbornane; 1,2-4-hydroxy-phenyl)ethane; 1,3-bis-(4-hydroxyphenyl)propenone; 4-hydroxyphenyl) sulfide; 4,4-bis-(4-hydroxyphenyl) pentanoic acid; 4,4-3,5-dimethyl-4-hydroxyphenyl) pentanoic acid; 2,2-bis-(4-hydroxyphenyl)acetic acid; 2,4′-dihydroxydiphenyl-methane; bis-(2-hydroxyphenyl)methane; bis-(4-hydroxy-phenyl)methane; bis-(4-hydroxy-5-nitrophenyl)methane; bis-(4-hydroxy-2,6-dimethyl-3-methoxyphenyl)methane; 1,1-bis-(4-hydroxyphenyl)ethane; 1,1-4-hydroxy-2-chlorophenyl)ethane; 2,2-bis-(4-hydroxyphenyl) propane (bisphenol-A); 1,1-bis-(4-hydroxyphenyl) propane; 2,2-bis-(3-chloro-4-hydroxyphenyl) propane; 2,2-bis-(3-bromo-4-hydroxyphenyl)propane; 2,2-bis-(4-hydroxy-3-methylphenyl)propane; 2,2-bis-(4-hydroxy-3-isopropylphenyl)propane; 2,2-bis-(3-t-butyl-4-hydroxyphenyl)propane; 2,2-bis-(3-phenyl-4-hydroxy-phenyl)propane; 2,2-3,5-dichloro-4-hydroxyphenyl)propane; 2,2-bis-(3,5-dibromo-4-hydroxyphenyl)propane; 2,2-bis-(3,5-dimethyl-4-hydroxy-phenyl)propane; 2,2-bis-(3-chloro-4-hydroxy-5-methylphenyl)propane; 2,2-bis-(3-bromo-4-hydroxy-5-methylphenyl)propane; 2,2-bis-(3-chloro-4-hydroxy-5-isopropylphenyl) propane; 2,2-bis-(3-bromo-4-hydroxy-5-isopropylphenyl)propane; 2,2-bis-(3-t-butyl-5-chloro-4-hydroxyphenyl)propane; 2,2-bis-(3-bromo-5-t-butyl-4-hydroxyphenyl)propane; 2,2-bis-(3-chloro-5-phenyl-4-hydroxyphenyl)propane; 2,2-bis-(3-bromo-5-phenyl-4-hydroxyphenyl)propane; 2,2-bis-(3,5-disopropyl-4-hydroxyphenyl)propane; 2,2-bis-(3,5-di-t-butyl-4-hydroxyphenyl)propane; 2,2-bis-(3,5-diphenyl-4-hydroxyphenyl) propane; 2,2-bis-(4-hydroxy-2,3,5,6-tetrachlorophenyl)propane; 2,2-bis-(4-hydroxy-2,3,5,6-tetrabromophenyl)propane; 2,2-bis-(4-hydroxy-2,3,5,6-tetramethylphenyl)propane; 2,2-bis-(2,6-dichloro-3,5-dimethyl-4-hydroxy-phenyl) propane; 2,2-bis-(2,6-dibromo-3,5-dimethyl-4-hydroxyphenyl)propane; 2,2-bis-(4-hydroxy-3-ethylphenyl)propane; 2,2-bis-(4-hydroxy-3,5-dimethylphenyl)propane; 2,2-bis-(3,5,3′,5′-tetrachloro-4,4′-dihydroxyphenyl)-propane; 1,1-bis-(4-hydroxyphenyl)cyclohexylmethane; 2,2-bis-(4-hydroxyphenyl)-1-phenylpropane; 1,1-bis-(4-hydroxyphenyl)cyclohexane; 1,1-bis-(3-chloro-4-hydroxyphenyl)cyclohexane; 1,1-bis-(3-bromo-4-hydroxyphenyl)cyclohexane; 1,1-bis-(4-hydroxy-3-methylphenyl)cyclohexane; 1,1-bis-(4-hydroxy-3-isopropylphenyl)cyclohexane; 1,1-bis-(3-t-butyl-4-hydroxyphenyl)cyclohexane; 1,1-bis-(3-phenyl-4-hydroxy-phenyl)cyclohexane; 1,1-bis-(3,5-dichloro-4-hydroxy-phenyl)cyclohexane; 1,1-bis-(3,5-dibromo-4-hydroxyphenyl)cyclohexane; 1,1-bis-(3,5-dimethyl-4-hydroxy-phenyl)cyclohexane; 1,1-bis-(3-chloro-4-hydroxy-5-methylphenyl)cyclohexane; 1,1-bis-(3-bromo-4-hydroxy-5-methylphenyl)cyclohexane; 1,1-bis-(3-chloro-4-hydroxy-5-isopropylphenyl)cyclohexane; 1,1-bis-(3-bromo-hydroxy-5-isopropylphenyl)cyclohexane; 1,1-bis-(3-t-butyl-5-chloro-4-hydroxyphenyl)cyclohexane; 1,1-bis-(3-bromo-5-t-butyl-4-hydroxyphenyl)cyclohexane; 1,1-bis-(3-chloro-5-phenyl-4-hydroxyphenyl)cyclohexane; 1,1-bis-(3-bromo-5-phenyl-4-hydroxyphenyl)cyclohexane; 1,1-bis-(3,5-disopropyl-4-hydroxyphenyl)cyclohexane; 1,1-bis-(3,5-di-t-butyl-4-hydroxyphenyl)cyclohexane; 1,1-bis-(3,5-diphenyl-4-hydroxyphenyl)cyclohexane; 1,1-bis-(4-hydroxy-2,3,5,6-tetrachlorophenyl)cyclohexane; 1,1-bis-(4-hydroxy-2,3,5,6-tetrabromo-phenyl)cyclohexane; 1,1-bis-(4-hydroxy-2,3,5,6-tetramethylphenyl)cyclohexane; 1,1-bis-(2,6-dichloro-3,5-dimethyl-4-hydroxyphenyl)cyclohexane; 1,1-bis-(2,6-dibromo-3,5-dimethyl-4-hydroxyphenyl)cyclohexane; 1,1-bis-(4-hydroxyphenyl) -3,3,5-trimethylcyclohexane; 1,1-bis-(3-chloro-4-hydroxyphenyl) -3,3,5-trimethylcyclohexane; 1,1-bis-(3-bromo-4-hydroxyphenyl) -3,3,5-trimethylcyclohexane; 1,1-bis-(4-hydroxy-3-methylphenyl) -3,3,5-trimethylcyclohexane; 1,1-bis-(4-hydroxy-3-isopropylphenyl) -3,3,5-trimethylcyclohexane; 1,1-bis-(3-t-butyl-4-hydroxyphenyl) -3,3,5-trimethylcyclohexane; 1,1-bis-(3-phenyl-4-hydroxyphenyl) 3,3,5-trimethylcyclohexane; 1,1-bis-(3,5-dichloro-4-hydroxyphenyl) -3,3,5-trimethylcyclohexane; 1,1-bis-(3,5-dibromo-4-hydroxyphenyl) -3,3,5-trimethylcyclohexane; 1,1-bis-(3,5-dimethyl-4-hydroxyphenyl) -3,3,5-trimethylcyclohexane; 1,1-bis-(3-chloro-4-hydroxy-5-methylphenyl) -3,3,5-trimethylcyclohexane; 1,1-bis-(3-bromo-4-hydroxy-5-methylphenyl) -3,3,5-trimethylcyclohexane; 1,1-bis-(3-chloro-4-hydroxy-5-isopropylphenyl) -3,3,5-trimethylcyclohexane; 1,1-bis-(3-bromo-4-hydroxy-5-isopropylphenyl) -3,3,5-trimethylcyclohexane; 1,1-bis-(3-t-butyl-5-chloro-4-hydroxyphenyl) -3,3,5-trimethylcyclohexane; 1,1-bis-(3-bromo-5-t-butyl-4-hydroxyphenyl) -3,3,5-trimethylcyclohexane; bis-(3-chloro-5-phenyl-4-hydroxyphenyl) -3,3,5-trimethylcyclohexane; 1,1-bis-(3-bromo-5-phenyl-4-hydroxyphenyl) -3,3,5-trimethylcyclohexane; 1,1-bis-(3,5-disopropyl-4-hydroxyphenyl) -3,3,5-trimethylcyclohexane; 1,1-bis-(3,5-di-t-butyl-4-hydroxyphenyl) -3,3,5-trimethylcyclohexane; 1,1-bis-(3,5-diphenyl-4-hydroxyphenyl) -3,3,5-trimethylcyclohexane; 1,1-bis-(4-hydroxy-2,3,5,6-tetrachlorophenyl) -3,3,5-trimethylcyclohexane; 1,1-bis-(4-hydroxy-2,3,5,6-tetrabromophenyl) -3,3,5-trimethylcyclohexane; 1,1-bis-(4-hydroxy-2,3,5,6-tetramethylphenyl) -3,3,5-trimethylcyclohexane; 1,1-bis-(2,6-dichloro-3,5-dimethyl-4-hydroxyphenyl) -3,3,5-trimethylcyclohexane; 1,1-bis-(2,6-dibromo-3,5-dimethyl-4-hydroxyphenyl) -3,3,5-trimethylcyclohexane; 4,4-bis-(4-hydroxyphenyl)heptane; 1,1-bis-(4-hydroxyphenyl)decane; 1,1-bis-(4-hydroxyphenyl)cyclododecane; 1,1-bis-(3,5-dimethyl-4-hydroxyphenyl)-cyclododecane; and bis-(4-hydroxyphenyl)methane.

The structural units of formula IV may be derived from aromatic dihalo compounds. Exemplary aromatic dihalo compounds include, but not limited to, 4,4′-dichlorodiphenyl sulfone, 4,4′-difluorodiphenyl sulfone, 1,4-bis(4-fluorophenylsulfone)benzene, 1,3-bis(4-fluorophenylsulfone)benzene, 1,4-bis(4-chlrophenylsulfone)benzene, and the like. The aromatic dihalo compounds may be similar to the ones described for the poly(arylether ketone)s or may be different.

The block copolymers may be made by the polycondensation of a dihydroxy endcapped poly(arylether sulfone) and a dihalo endcapped poly(arylether ketone). The poly(arylether sulfone) blocks having dihydroxy end groups may be prepared by polycondensation of a slight molar excess of dihydroxyaryl monomers with dihalodiarylsulfones or polycondensation of dihalodiarylsulfone monomers, such as dichlorodiphenylsulfone, with a slight molar excess of dihydroxydiarylsulfones, such as dihydroxydiphenylsulfone. The amount of molar excess to be used in the reaction mixture depends on the desired molecular weight of the block, reaction temperature, and the like, and can be determined without undue experimentation by one of ordinary skill in the art. Examples of suitable dihalodiphenyl sulfones include 4,4′-dichlorodiphenylsulfone and 4,4′-difluorodiphenylsulfone. In a similar manner, poly(arylether ketone)s with dihalo end groups may be prepared. In this instance, however, a slight molar excess of the dihalo compounds is reacted with the dihydroxy compounds. Suitable dihydroxy compounds include those containing aromatic keto groups. In one exemplary embodiment, the aromatic dihydroxy compound is 1,4-bis(4-hydroxybenzoyl)benzene.

The poly(arylether ketone) blocks also include structural units derived from

dihydroxy compounds having

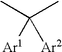

linkages. Exemplary compounds having such linkages include 4,4′-dihydroxytetraphenylmethane, and 9,9-bis(4-hydroxyphenyl)fluorene.

The weight average molecular weight of the polyaryletherketone blocks ranges from about 2000 Daltons to about 15000 Daltons. The weight average molecular weight of the polyethersulfone blocks ranges from about 2000 Daltons to about 20000 Daltons. In some embodiments, the molecular weight of the final polymer may range from about 20000 Daltons to about 100000 Daltons. Total molecular weight of the sulfonated block copolymers is typically not critical, although higher molecular weights, that is, 100,000-150,000 Daltons, may be desirable in some embodiments. Weight average molecular weights may be determined by any techniques known in the art. Such techniques include light scattering, gel permeation chromatography (GPC), and the like. It will be apparent to those skilled in the art that different molecular weight determination techniques give rise to different averages of molecular weight. Thus, gel permaeation chromatography provides both number-average as well as weight-average molecular weight, while light scattering techniques gives rise to weight average molecular weights. In some embodiments, GPC is performed using a suitable mobile phase such as dimethyl acetamide, and the molecular weight estimated based on known standards such as polystyrene, polyethylene oxide, and the like. In some particular embodiments, gel permeation chromatography in N,N-dimethylacetamide/LiBr using polystyrene, polyethersulfone, or polyethylene glycol standards is used.

The block copolymers may be prepared by processes known in the art. These include nucleophilic displacement of stoichiometric quantities of bisphenolate salts with activated aromatic dihalides in polar aprotic solvents. In particular, the block copolymers may be synthesized by nucleophilic aromatic substitution using potassium carbonate in polar solvents such as dimethylsulfoxide (DMSO), dimethyl acetamide (DMAc), dimethyl formamide (DMF), anisole, veratrole, and the like.

The polymers may also be prepared using phase transfer-catalyzed nucleophilic displacement of bisphenols with dihaloaryl monomers. Suitable phase transfer catalysts include hexaalkylguanidinium salts and bis-guanidinium salts. Typically the phase transfer catalyst comprises an anionic species such as halide, mesylate, tosylate, tetrafluoroborate, or acetate as the charge-balancing counterion(s). Suitable guanidinium salts include those disclosed in U.S. Pat. No. 5,132,423; U.S. Pat. No. 5,116,975 and U.S. Pat. No. 5,081,298. Other suitable phase transfer catalysts include p-dialkylamino-pyridinium salts, bis-dialkylaminopyridinium salts, bis-quaternary ammonium salts, bis-quaternary phosphonium salts, and phosphazenium salts. Suitable bis-quaternary ammonium and phosphonium salts are disclosed in U.S. Pat. No. 4,554,357. Suitable aminopyridinium salts are disclosed in U.S. Pat. No. 4,460,778; U.S. Pat. No. 4,513,141 and U.S. Pat. No. 4,681,949. Suitable phosphazenium salts are disclosed in US 2006/0069291. Additionally, in certain embodiments, the quaternary ammonium and phosphonium salts disclosed in U.S. Pat. No. 4,273,712 may also be used.

Sulfonation is achieved by reacting the polymer with a suitable sulfonating agent, such as SO₃, ClSO₃H, Me₃SiSO₃Cl, fuming or concentrated H₂SO₄, and the like. Solvents may be used or it may be conducted as a neat reaction.

The monomers for the block copolymers are chosen such that the sulfonation occurs at the pendant aromatic groups. Presence of a C(Ar)₂ linkages results in sulfonation at the Ar groups as they are more conducive for electrophilic substitution reactions, such as sulfonation, as compared to —O—Ar—C(O)— linkages, —O—Ar—C(CF₃)₂— linkages, or —O—Ar—S(O)₂-linkages. Presence of O—Ar—O linkages may result in the competing sulfonation of this unit along with the C(Ar)₂ as well, which is undesirable. Thus, by the choice of monomers and appropriate reaction conditions, all the sulfonate groups of the block copolymers are made available on the poly(arylether ketone) blocks.

The sulfonated block copolymers typically contain from about 20 to about 80 mol % sulfonation, particularly from about 30 to about 60 mol % sulfonation. The term “mol % sulfonation” means mol % of the structural units derived from a ketone monomer and containing at least one sulfonate group, with respect to the total moles of structural units derived from ketone. That is, mol % sulfonation means the mol % of the structural units of formula I, with respect to the total moles of structural units of formula I and structural units of formula II, where the only structural units included in the block copolymers that are derived from ketone monomers are the structural units of formula I and structural units of formula II.

In particular, the individual blocks of poly(arylether ketone)s and the poly(arylether sulfone)s and the block copolymers, may be synthesized by the polymerization reaction of one or more bisphenol compounds such as bisphenols or bisphenolate salts, particularly those containing pendant aromatic groups, with a dihalobenzophenone in a polar aprotic solvent, such as N,N-dimethylacetamide (DMAc), and an azeotroping solvent, such as toluene, under refluxing conditions. The reaction is generally catalyzed by a base, preferably an inorganic base such as potassium carbonate, potassium hydroxide or cesium fluoride. Generally two equivalents of the base are used with respect to the bisphenol.

In separate embodiments, the present invention also relates to membranes, especially proton exchange or polymer electrolyte membranes, that include any of the sulfonated block copolymers according to the present invention, and to fuel cells that include the membranes.

Membranes may be prepared by casting films from a solution of the block copolymers of the invention in a suitable solvent. Solutions may be filtered and degassed prior to film casting. Films may be cast onto a substrate of choice, which may be any flat surface that shows no interaction towards any or all of the components of the solution. Suitable substrates may include, but not limited to, glass, metal and the like.

Proton conductivity of the membranes may be determined by standard techniques known in the art. Commercially available instruments may be used for evaluating membranes for their proton conductivity efficacy. This generally involves the measurement of the impedance generated by the membrane at various humidity levels and temperatures. In some instances, polymers of the invention gave conductivity values of greater than 0.05 S/cm under the testing conditions.

### Definitions

In the context of the present invention, alkyl is intended to include linear, branched, or cyclic hydrocarbon structures and combinations thereof, including lower alkyl and higher alkyl. Preferred alkyl groups are those of C₂₀ or below. Lower alkyl refers to alkyl groups of from 1 to 6 carbon atoms, preferably from 1 to 4 carbon atoms, and includes methyl, ethyl, n-propyl, isopropyl, and n-, s- and t-butyl. Higher alkyl refers to alkyl groups having seven or more carbon atoms, preferably 7-20 carbon atoms, and includes n-, s- and t-heptyl, octyl, and dodecyl. Cycloalkyl is a subset of alkyl and includes cyclic hydrocarbon groups of from 3 to 8 carbon atoms. Examples of cycloalkyl groups include cyclopropyl, cyclobutyl, cyclopentyl, and norbornyl.

Aryl and heteroaryl mean a 5- or 6-membered aromatic or heteroaromatic ring containing 0-4 heteroatoms selected from nitrogen, oxygen or sulfur; a bicyclic 9- or 10-membered aromatic or heteroaromatic ring system containing 0-4 heteroatoms selected from nitrogen, oxygen or sulfur; or a tricyclic 13- or 14-membered aromatic or heteroaromatic ring system containing 0-4 heteroatoms selected from nitrogen, oxygen or sulfur. The aromatic 6- to 14-membered carbocyclic rings include, for example, benzene, naphthalene, indane, tetralin, and fluorene; and the 5- to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole.

Arylalkyl means an alkyl residue attached to an aryl ring. Examples are benzyl and phenethyl. Heteroarylalkyl means an alkyl residue attached to a heteroaryl ring. Examples include pyridinylmethyl and pyrimidinylethyl. Alkylaryl means an aryl residue having one or more alkyl groups attached thereto. Examples are tolyl and mesityl.

Alkoxy or alkoxyl refers to groups of from 1 to 8 carbon atoms of a straight, branched, cyclic configuration and combinations thereof attached to the parent structure through an oxygen. Examples include methoxy, ethoxy, propoxy, isopropoxy, cyclopropyloxy, and cyclohexyloxy. Lower alkoxy refers to groups containing one to four carbons.

Acyl refers to groups of from 1 to 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, and benzyloxycarbonyl. Lower-acyl refers to groups containing one to four carbons.

Heterocycle means a cycloalkyl or aryl residue in which one to four of the carbons is replaced by a heteroatom such as oxygen, nitrogen or sulfur. Examples of heterocycles that fall within the scope of the invention include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substitutent), tetrazole, morpholine, thiazole, pyridine, pyridazine, pyrimidine, thiophene, furan, oxazole, oxazoline, isoxazole, dioxane, and tetrahydrofuran, triazole, benzotriazole, and triazine.

Substituted refers to structural units, including, but not limited to, alkyl, alkylaryl, aryl, arylalkyl, and heteroaryl, wherein up to three H atoms of the residue are replaced with lower alkyl, substituted alkyl, aryl, substituted aryl, haloalkyl, alkoxy, carbonyl, carboxy, carboxalkoxy, carboxamido, acyloxy, amidino, nitro, halo, hydroxy, OCH(COOH)₂, cyano, primary amino, secondary amino, acylamino, alkylthio, sulfoxide, sulfone, phenyl, benzyl, phenoxy, benzyloxy, heteroaryl, or heteroaryloxy; each of said phenyl, benzyl, phenoxy, benzyloxy, heteroaryl, and heteroaryloxy is optionally substituted with 1-3 substitutents selected from lower alkyl, alkenyl, alkynyl, halogen, hydroxy, haloalkyl, alkoxy, cyano, phenyl, benzyl, benzyloxy, carboxamido, heteroaryl, heteroaryloxy, nitro or —NRR (wherein R is independently H, lower alkyl or cycloalkyl, and —RR may be fused to form a cyclic ring with nitrogen).

Haloalkyl refers to an alkyl residue, wherein one or more H atoms are replaced by halogen atoms; the term haloalkyl includes perhaloalkyl. Examples of haloalkyl groups that fall within the scope of the invention include CH₂F, CHF₂, and CF₃.

Any numerical values recited herein include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80, more preferably from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

### EXAMPLES

Block poly(aryl ether)s are synthesized in a three-step process. This is achieved by potassium carbonate mediated direct aromatic nucleophilic substitution polycondensation of phenoxides and aromatic halides to form oligomer poly(aryl ether ketone)s and oligomer poly(aryl ether sulfone)s. Subsequently, polycondensation of oligomer poly(aryl ether ketone)s and oligomer poly(aryl ether sulfone)s yields the block copolymers. Copolymerization proceeds quantitatively to yield high molecular weight polymers in dimethyl acetamide (DMAc) at 155-165° C. as shown in Table 1. Sulfonated polyetherketones-block-polyethersulfones are prepared by sulfonation of polyetherketones-block-polyethersulfones using concentrated sulfuric acid at room temperature for the desired time. Strong and flexible films are successfully cast from the solution of sulfonated polyetherketones-block-polyethersulfones in DMAc.

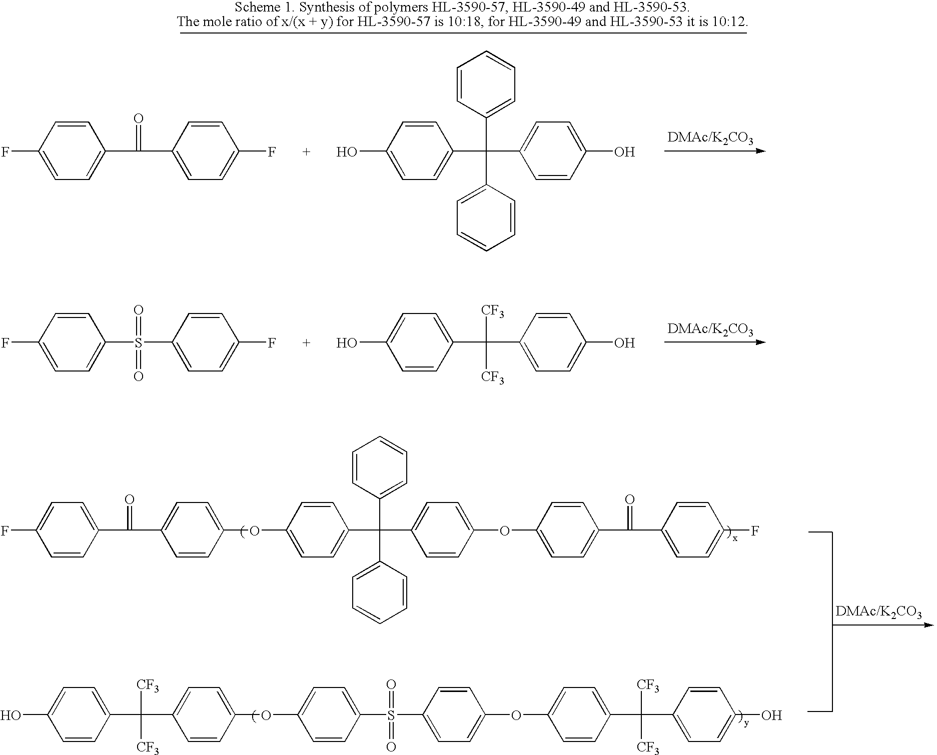

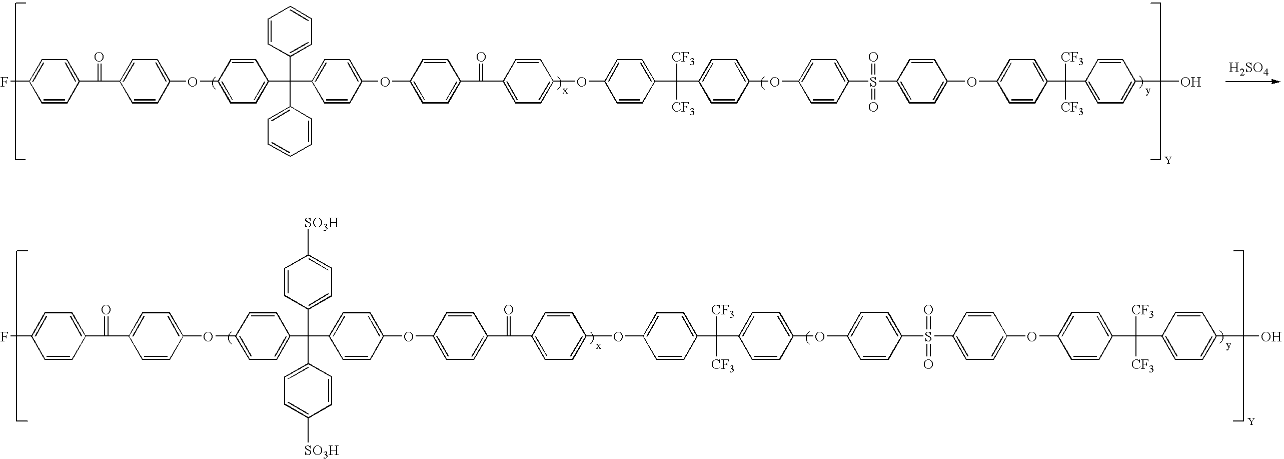

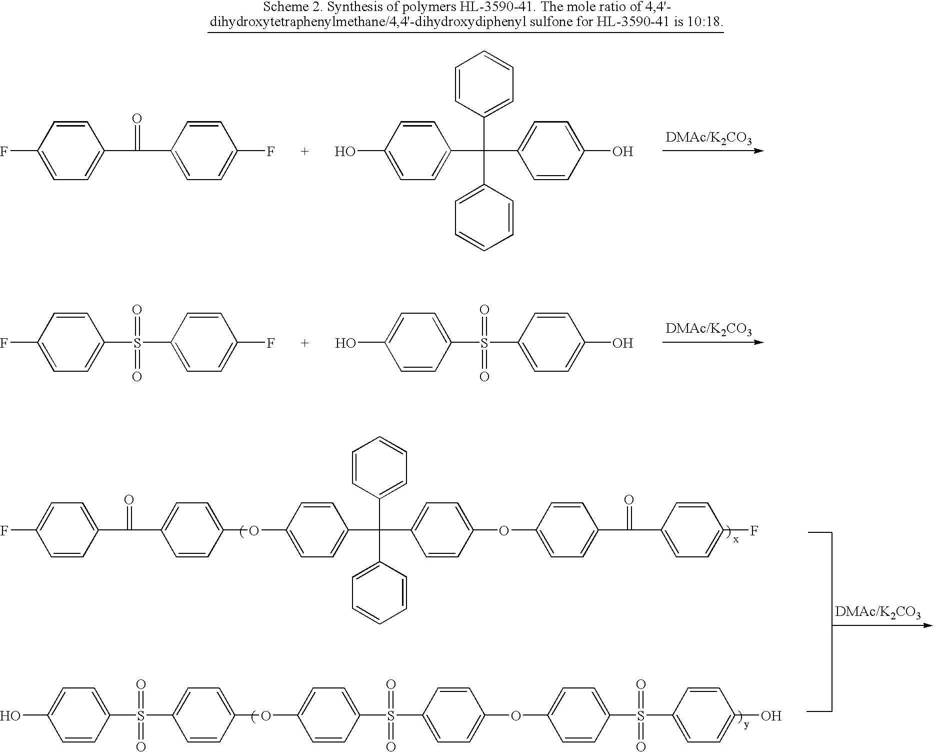

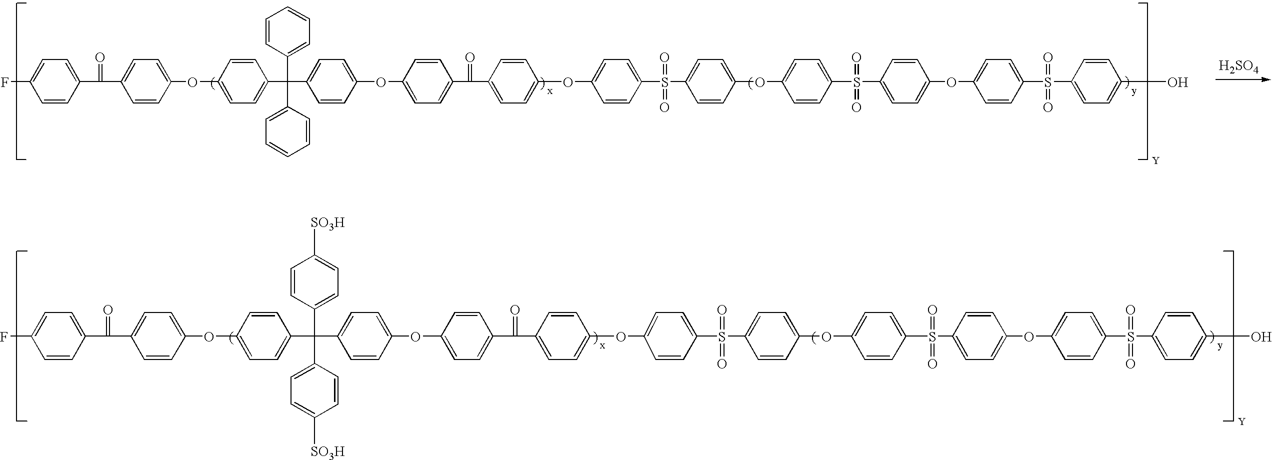

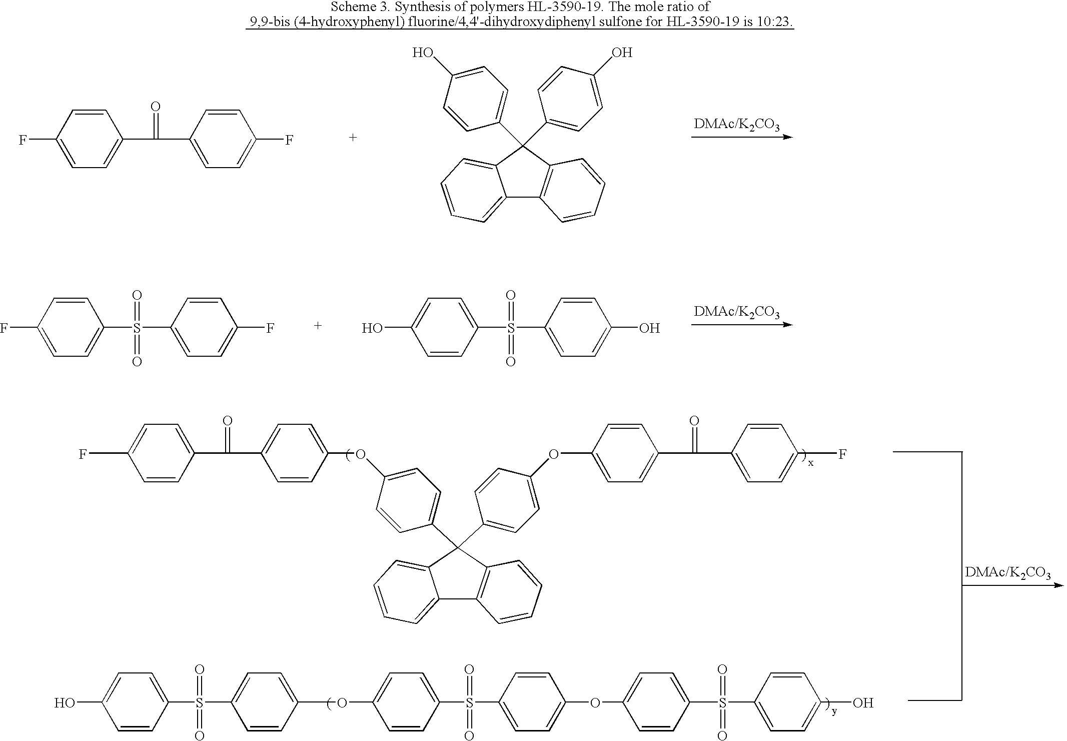

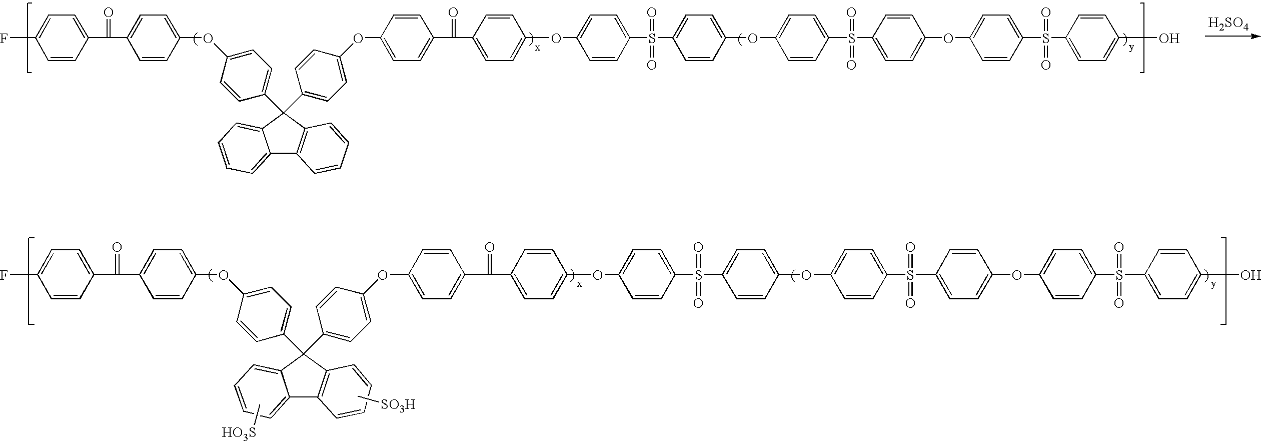

General Procedures: Potassium carbonate is dried in oven at 140° C. before use, and all the other chemicals are used as received. Molecular weights of all polymers are measured by Size Exclusion Chromatography (SEC) as described: Non-sulfonated polymers are dissolved in chloroform and are analyzed on a Polymer labs Mixed C column, eluting with 3% isopropanol/chloroform at a flow rate of 0.8 milliliters per minute (mL/min), and using UV detection. Sulfonated polymers are dissolved in 0.05 molar (M) LiBr/DMAc, and analyzed on a Polymer labs Mixed C column, eluting with 0.05 M LiBr/DMAc at 0.08 mL/min, and using refractive index detection. Molecular weights are calculated using Turbogel software, relative to polystyrene standards for the non-sulfonated materials, or relative to secondary polyethersulfone standards for the sulfonated materials. The secondary standards' molecular weights are measured in chloroform (relative to polystyrene), and then used to calibrate the analysis in LiBr/DMAc. Nafion 117 was purchased from Aldrich Chemical Company

### Example 1

### Synthesis of Polyetherketone (PEK)

4,4′-fluorobenzophenone (2.6184 grams (g), 12 millimole (mmol)), 4,4′-dihydroxytetraphenylmethane (3.5243 g, 10 mmol), dry DMAc (30 mL) and potassium carbonate (1.94 g, 14 mmol) are added into a three neck round bottom flask equipped with a mechanical stirrer and a nitrogen inlet. Toluene (15 mL) is used as an azeotropic agent. The reaction mixture is heated at 155° C. for 4 hours (h), and then at 165° C. for 18 h. The polymer solution becomes viscous and is then cooled to room temperature under nitrogen for the next step reaction.

### Example 2

### Synthesis of Polyethersulfone (PES)

4,4′-difluorodiphenyl sulfone (4.068 g, 16 mmol), 4,4′-(hexafluoroisopropylidene) diphenol (6.0521 g, 18 mmol) (to give a mole ratio between the monomers 4,4′-dihydroxytetraphenylmethane/4,4′-(hexafluoroisopropylidene) diphenol of 10:12), dry DMAc (40 mL) and potassium carbonate (3.72 g, 26.7 mmol) are added into a three neck round bottom flask that is equipped with a mechanical stirrer and a nitrogen inlet. Toluene (20 mL) is used as an azeotropic agent. The reaction mixture is heated at 155° C. for 4 h, and then at 165° C. for 18 h. The polymer solution becomes viscous and is then cooled to room temperature under nitrogen for the next step reaction.

### Example 3

### Synthesis of PEK-Block-PES

Polymer solution of PEK prepared above is transferred to a three neck round bottom flask containing polymer solution of PES at room temperature under nitrogen. The mixture of two polymers is heated to 165° C. for 20 h under nitrogen. The polymer was precipitated into a 1:1 v/v mixture of water and methanol while blending. The precipitated polymer is collected by filtration, and is washed extensively with de-ionized water and ethanol to remove salt, and is finally dried in a vacuum oven overnight.

### Example 4

### Synthesis of Sulfonated PEK-Block-PES HL-3590-57

Sulfonation is carried out by dissolving the above PEK-block-PES (1.5 g) in concentrated sulfuric acid (20 mL), and stirred for 3 hours at room temperature. After the elapsed time, the mixture is poured into ice water. The sulfonated polymer is collected by filtration, and is washed with de-ionized water until the pH of the rinse water is between 6-7. The polymer is then collected and dried at room temperature for 2 days followed by drying in a vacuum oven at 80° C. for 24 h.

### Example 5

### Synthesis of Polymer HL-3590-53

The procedures described for the synthesis of polymer HL-3590-57 is followed here as well, except the molar ratio between the monomers 4,4′-dihydroxytetraphenylmethane/4,4′-(hexafluoroisopropylidene) diphenol is maintained at 10:18.

### Example 6

### Synthesis of Polymer HL-3590-49

The procedures described for the synthesis of polymer HL-3590-53 is followed here as well, except the sulfonation is allowed to proceed for 16 hours.

### Example 7

### Synthesis of Polymer HL-3590-41

The procedures delineated for polymer BL3590-49 is followed here, except 4,4′-(hexafluoroisopropylidene) diphenol is replaced with 4,4-dihydroxy diphenyl sulfone to produce a sulfonated PEK-block-PES.

### Example 8

### Synthesis of Polymer HL-3590-18

The procedures delineated for polymer HL3590-49 is followed here, except 4,4′-dihydroxytetraphenylmethane is replaced with 9,9-bis(4-hydroxyphenyl)fluorene, while maintaining the mole ratio between 9,9-bis(4-hydroxyphenyl)fluorene/4,4′-dihydroxydiphenyl sulfone at 10:23, and carrying out the sulfonation for 16 hours to produce a sulfonated PEK-block-PES.

### Example 9

### Synthesis of Polymer HL-3590-19

The procedures delineated for polymer HL3590-18 is followed here, except that the sulfonation is allowed to proceed for 16 hours to produce a sulfonated PEK-block-PES.

Molecular Weights, Daltons

| | | | | Sulfonated |
|---|---|---|---|---|
| | | | Block | Block |
| Polymer | PEK | PES | Copolymer | Copolymer |
| HL-3590-57 | 6600 | 8300 | 101,000 | N/A |
| HL-3590-49 | 6600 | 12,600 | 70,000 | N/A |
| HL-3590-53 | 6600 | 12,600 | 70,000 | N/A |
| HL-3590-41 | 6500 | N/A | 49,800 | N/A |
| HL-3590-18 | 7800 | N/A | 39,000 | 49,400 |
| HL-3590-19 | 7800 | N/A | 39,000 | 48,900 |

### Example 10

### Membrane Preparation

Dried sulfonated block polymer (1 g) is dissolved in DMSO (4 mL), then the solution is filtered using a glass fritted filter funnel under vacuum. The film is cast from polymer solution onto a glass plate using film applicator at room temperature, and is then dried at room temperature for 1 day, followed by drying at 80° C. under vacuum overnight. The thickness of the film is about 0.02 millimeters (mm).

### Example 11

### Membrane Proton Conductivity Measurement

The proton conductivity of the polymer membranes is determined by 4-electrode impedance measurements at various temperatures and relative humidities. Measurements use a PARSTAT® Model 2263 impedance analyzer, sold by Princeton Applied Research with PowerSINE software, using a signal amplitude that ranges from 5 to 50 millivolts (mV) and frequencies ranging from 2 Hz to 2 MHz. The sample dimensions varies between samples, with a typical sample being 1.5 centimeters (cm)×2.5 cm and having a thickness ranging from 20 microns (μm) to 100 μm. Typical membranes are 25-50 μm in thickness.

TABLE 5
Proton conductivity of sulfonated block poly(aryl ether) at
different experimental condition.

| | % | | | | | | |
|---|---|---|---|---|---|---|---|
| | Relative | Conductivity, Siemens per centimeter (S/cm) | | | | | |
| Temp | Humid- | HL- | HL- | HL- | HL- | HL- | HL- |
| [° C.] | ity | 3590-49 | 3590-53 | 3590-57 | 3590-41 | 3590-18 | 3590-19 |
| 20 | 100 | 0.0109 | 0.0035 | 0.0020 | 0.0057 | 0.0014 | 0.0044 |
| 60 | 50 | 0.0003 | <0.0001 | 0.0001 | <0.0001 | <0.0001 | <0.0001 |
| 80 | 25 | <0.0001 | <0.0001 | <0.0001 | <0.0001 | <0.0001 | <0.0001 |
| 80 | 50 | 0.0003 | <0.0001 | 0.0001 | 0.0002 | <0.0001 | 0.0001 |
| 80 | 75 | 0.0011 | 0.0010 | 0.0024 | 0.0050 | 0.0013 | 0.0092 |
| 80 | 100 | 0.0228 | 0.0288 | 0.0504 | 0.1703 | 0.0189 | 0.0172 |
| 100 | 50 | 0.0008 | 0.0002 | 0.0007 | 0.0005 | 0.0013 | 0.0011 |
| 100 | 75 | 0.0067 | 0.0011 | 0.0077 | 0.0049 | 0.0034 | 0.0033 |
| 120 | 50 | 0.0008 | <0.0001 | 0.0004 | 0.0007 | 0.0005 | 0.0005 |

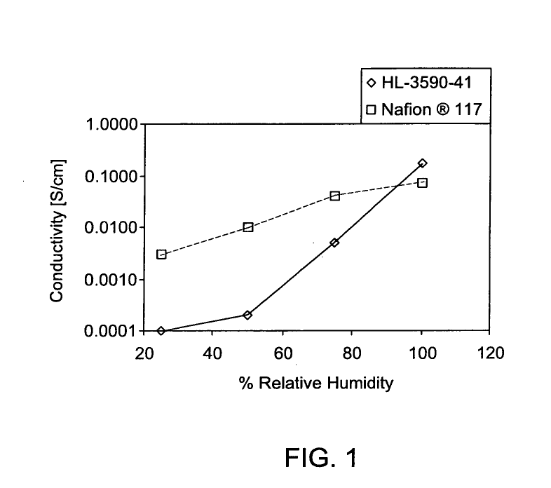

FIG. 1 shows the comparison of conductivity of polymer HL-3590-41 with Nafion® 117 at 80° C. The conductivity of HL-3590-41 is higher than Nafion® 117 at 100% relatively humidity, while lower than Nafion® 117 at low relative humidity.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A sulfonated block copolymer comprising unsulfonated polyethersulfone blocks and sulfonated polyaryletherketone blocks comprising structural units of formula I 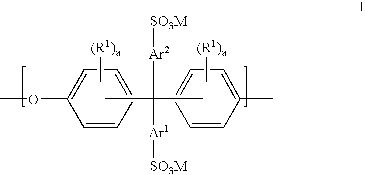
wherein R¹ is C₁-C₁₀ alkyl, C₃-C₁₂ cycloalkyl, C₆-C₁₄ aryl, allyl, alkenyl, alkoxy, halo, or cyano;
Ar¹ and Ar² are each independently C₆-C₂₀ aromatic radicals, or Ar¹ and Ar², taken together with an intervening carbon atom, form a bicyclic C₆-C₂₀ aromatic radical or a tricyclic C₆-C₂₀ aromatic radical;
M is H, a metal cation, a non-metallic inorganic cation, an organic cation or a mixture thereof; and
a is 0 or an integer from 1 to 4.

2. A sulfonated block copolymer according to claim 1, wherein the sulfonated polyaryletherketone blocks further comprise structural units of formula II 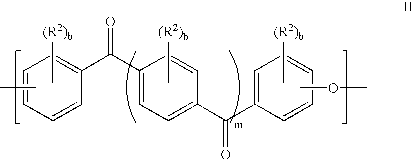
wherein R² is C₁-C₁₀ alkyl, C₃-C₁₂ cycloalkyl, C₆-C₁₄ aryl, allyl, alkenyl, alkoxy, halo, or cyano;
b is 0 or an integer from 1 to 4; and
m is 0 or 1.

3. A sulfonated block copolymer according to claim 1, wherein the unsulfonated polyethersulfone blocks comprise structural units of formula III 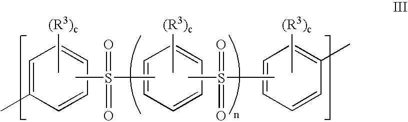
wherein R³ is C₁-C₁₀ alkyl, C₃-C₁₂ cycloalkyl, C₆-C₁₄ aryl, allyl, alkenyl, alkoxy, halo, or cyano;
c is 0 or an integer from 1 to 4; and
n is 0 or 1.

4. A sulfonated block copolymer according to claim 1, wherein the unsulfonated polyethersulfone blocks comprise structural units of formula IV 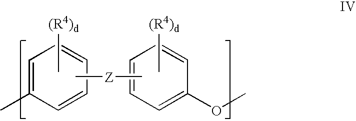
wherein R⁴ is C₁-C₁₀ alkyl, C₃-C₁₂ cycloalkyl, C₆-C₁₄ aryl, allyl, alkenyl, alkoxy, halo, or cyano;
Z is a direct bond, O, S, (CH₂)ᵣ, (CF₂)ᵣ, C(CH₃)₂, C(CF₃)₂, or SO₂;
d is 0 or an integer from 1 to 4; and
r is an integer from 1 to 5.

5. A sulfonated block copolymer according to claim 1, wherein the structural units of formula I are

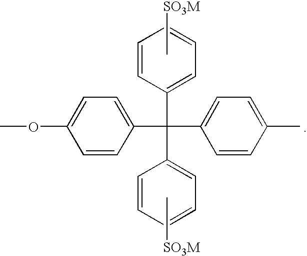

6. A sulfonated block copolymer according to claim 1, wherein the structural units of formula I are

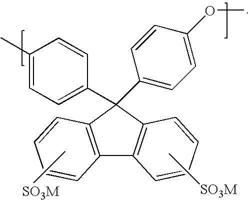

7. A sulfonated block copolymer according to claim 4, wherein Z is C(CF₃)₂.

8. A sulfonated block copolymer according to claim 4, wherein Z is SO₂.

9. A sulfonated block copolymer according to claim 1, comprising from about 20 mol % to about 80 mol % of the structural units of formula I.

10. A sulfonated block copolymer according to claim 1, comprising from about 30 mol % to about 60 mol % of the structural units of formula I.

11. A sulfonated block copolymer according to claim 1, wherein the molecular weight of the sulfonated polyaryletherketone blocks ranges from about 2000 Daltons to about 15000 Daltons.

12. A sulfonated block copolymer according to claim 1, wherein the molecular weight of the polyethersulfone blocks ranges from about 2000 Daltons to about 20000 Daltons.

13. A sulfonated block copolymer comprising sulfonated polyaryletherketone blocks comprising structural units of formula V 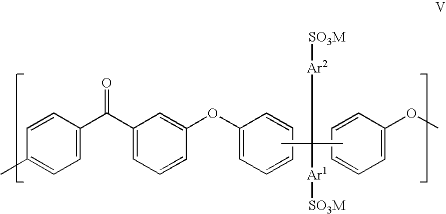
and unsulfonated polyethersulfone blocks comprising structural units of formula VI 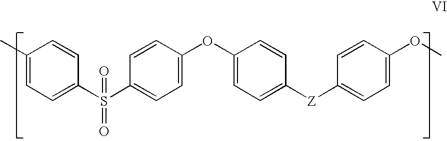
Ar¹ and Ar² are each independently C₆-C₂₀ aromatic radicals, or Ar¹ and Ar², taken together with an intervening carbon atom, form a bicyclic C₆-C₂₀ aromatic radical or a tricyclic C₆-C₂₀ aromatic radical;
M is H, a metal cation, a non-metallic inorganic cation, an organic cation or a mixture thereof;
Z is a direct bond, O, S, (CH₂)ᵣ; (CF₂)ᵣ, C(CH₃)₂, C(CF₃)₂, or SO₂;
r is an integer from 1 to 5.

14. A sulfonated block copolymer according to claim 13, wherein Z is C(CF₃)₂.

15. A sulfonated block copolymer according to claim 13, wherein Z is SO₂.

16. A sulfonated block copolymer according to claim 13, wherein the structural units of formula V are

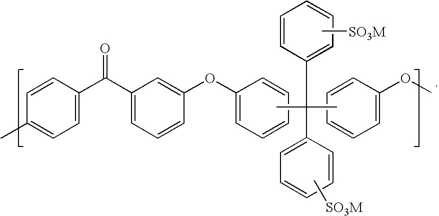

17. A sulfonated block copolymer according to claim 13, wherein the structural units of formula V are

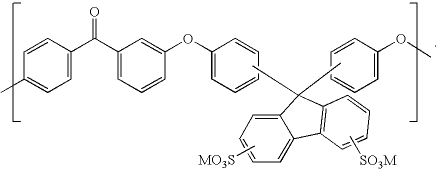

18. A membrane comprising a sulfonated block copolymer according to claim 1.

19. A membrane-electrode assembly comprising a membrane according to claim 18.

20. A fuel cell comprising a membrane according to claim 18.

21. A membrane comprising a sulfonated block copolymer according to claim 13.

22. A membrane-electrode assembly comprising a membrane according to claim 21.

23. A fuel cell comprising a membrane according to claim 21.
